(19) United States
(12) Patent Application Publication
(10) Pub. No.: US 2024/0108396 A1
(43) Pub. Date: Apr. 4, 2024

(54) V Nose for Smoke Evacuation

(71) Applicant: Robert Lee Bromley, El Paso, TX (US)

(72) Inventor: Robert Lee Bromley, El Paso, TX (US)

(21) Appl. No.: 17/803,665

(22) Filed: Sep. 29, 2022

(51) Int. Cl. A61B 18/14

(52) U.S. Cl. CPC A61B 18/1402; A61B 2218/008

(57) ABSTRACT

A novel smoke evacuation distal end having a distal port axially rotatable or fixedly attached to an energy assisted surgical device with at least one non-obstructing cutout to allow for improved surgical visibility while maintaining smoke suction performance.

### BACKGROUND OF INVENTION

Prior art energy assisted surgical devices were used in procedures involving an ‘energy source means’ to create physiological tissue effects resulting in smoke plumes. Inhaled smoke plumes by operating room personnel continue to give concerns of potentially harmful health effects. These concerns raise awareness for a smoke evacuation means either being attached to, or integrated into, said energy assisted surgical devices to evacuate smoke plumes. Prior art smoke evacuation devices have distal port openings which are generally circular and larger than the ‘energy source means’, when compared to a non-smoke evacuating surgical device; thereby, reducing the surgeons' line of sight when operated at acute angles. The typical smoke evacuating distal port opening is co-planar, but not limited to co-planar, to a plane perpendicular to the surgical devices' longitudinal axis. A perpendicular or near perpendicular distal end opening is efficient for effective smoke evacuation, the continuous round horizon line of the distal port obstructs optimal viewing from acute angle surgeon lines of sight. Prior art distal ports use various size diameters and tapered shapes to find a compromise between the optimal smoke evacuation distal port opening and optimal surgeon line of sight. For instance, a cylindrical distal port would have a circular horizon line of cylindrical, conical, or multi-faceted edges typically, concentric with the ‘energy source means’ which are often tapered to reduce visual obstruction at the distal port opening. Taper shaped prior art distal ports offer improved visibility but reduce the smoke evacuation suction flow due to smaller distal port openings. The distal port openings while having various shapes to improve visibility, create suction flow restrictions caused by a reduced cross-sectional area of the distal port opening. Translucent distal ports have minimal tapers to optimize suction flow but create visual distortions caused by the translucent cylindrical shape and material. Visual distortions prevent precise placement of the ‘energy source means’ onto the ‘energy point of contact’. Precise placement is critical for physiological effects such as surgical incisions, surgical sealing and/or tissue oblation, which is highly dependent on a clear, undistorted view of the patient ‘energy point of contact’.

Acute angles as defined in this embodiment as the inclusive angle between the surgical device longitudinal axis in the surgeons' hand to the surgeons' line of sight being less than 45 degrees. Acute angles due to limited surgical space create greater visual obstructions than obtuse angles. A large distal port diameter has a distal port opening conducive to smoke evacuation flow compared to smaller distal port diameters; however, the more acute the viewing angle due to the surgeons' physical constraints accessing the minimal opening of the patient body cavity; results in limited surgeon visibility of the ‘energy source means’ at the ‘energy point of contact’. Prior art for smoke evacuation energy assisted surgical devices such as, but not limited to, plasma, radiation, conduction, ionized gas, laser, ultrasonic, or RFI energy, have a distal opening which is typically concentric and round to the ‘energy source means’.

An analogy for prior art surgeon visual obstruction would be to consider a surgeon wearing a head mounted light source nearly parallel to the surgeons' line of sight which illuminates the visibility of the patient tissue and the targeted ‘energy point of contact’ by using the ‘energy source means’. In prior art, as the surgeon mounted light beam moves from an obtuse angle narrowing to create a more acute angle between the surgeons' line of sight and the longitudinal axis of the smoke evacuating surgical device, the distal end port horizon has a shadow encroaching upon and beginning to obstruct the visibility of the ‘energy point of contact’ and the ‘energy source means’. As the acute angle becomes smaller said ‘energy point of contact’ and ‘energy source means’ are completely in shadow. The surgeons' vison becomes obstructed by the distal port horizon line.

Common hand positions holding a smoke evacuating surgical device use the index finger and thumb to apply force to grip said energy assisted device by those skilled in the art of energy-based surgery. Once the surgical device is placed in the surgeons' hand various anatomical targets of and within a patient's body may cause the surgeons' forearm position and wrist rotation to create an acute angle formed between the longitudinal axis of the energy assisted device to the surgeons' line of sight. A non-rotatable distal port having a non-obstructing cutout to the distal port opening would require the smoke evacuating surgical device to rotate about the longitudinal axis to providing visual alignment of the surgeon's line of sight and the ‘energy source means’ and ‘energy point of contact’; however, due to anatomical constraints of wrist adaptations and finger dexterity to operating hand controlled buttons, selective alignment of the non-obstructing cutout to the surgeons' line of sight may not possible; therefore, in this embodiment, narrow acute visual angles of the distal port may employ a rotatable, but not limited to rotatable, means to allow alignment of the non-obstructing cutout to the surgeon's line of sight.

In this novel invention a more visually accommodating distal port having at least one, but not limited to one, adjacent side feature near the distal port opening to interrupt the horizon line and improve surgeon visibility while optimizing smoke evacuation flow for clear unobstructed views previously not available by prior art distal ports. Said adjacent side features may be material removal i.e., cutouts or material relocation or surface changes altering the horizon line.

The following illustrations show a hand-held electrosurgical pencil; however, those skilled in the art of energy assisted surgical devices can easily adapt the visual advantages of this novel distal port when used on other smoke evacuating energy assisted surgical devices such as laser, ultrasound, ionization, convection, RFI, laparoscopic, endoscopic, and robotic energy assisted procedures to name a few. In addition the drawings depict the ‘energy source means’ as an active electrode protruding from the distal end and is only for representative purposes of defining a working distance from the smoke evacuating device to the ‘energy point of contact’, those skilled in the art can easily understand the working distance needed when using an ‘energy source means’ such as a laser, ionized gas, or ultrasonic energy in substitution of the active electrode.

### SUMMARY OF THE INVENTION

A novel invention of a V shaped distal port on a smoke evacuating energy assisted surgical device with a distal port opening forming at least one non-obstructing cutout to allow a surgeons' line of sight, visibility of the ‘energy point of contact’ and ‘energy source means’ at acute angles. At least one distal port non-obstructing cutout creates localized horizon line transitions which can be, but not limited to, axially symmetrical or axially asymmetrical cutouts by, but not limited to, surface changes or material removed cutouts. Various line of sight angles may require a rotatable, but not limited to a rotatable, distal port with at least one non-obstructing cutout located around the surgical devices' longitudinal axis to align said non-obstructing cutout to various acute angle positions of the surgeon's line of sight providing unobstructed visibility of the ‘energy source means’ and ‘energy point of contact’.

### BRIEF DESCRIPTION OF DRAWINGS

FIG. 1 is a back view of a surgeon holding a smoke evacuating surgical device oriented with the longitudinal axis in relation to the surgeons' line of sight forming an obtuse angle.

FIG. 2 is a top view of a surgeon holding a smoke evacuating surgical device oriented with the longitudinal axis in relation to the surgeons' line of sight forming an acute angle.

FIG. 3 is a top view of a surgeon holding a smoke evacuating surgical device forming an acute angle having the distal port rotated to visually align the distal port non-obstructing cutout.

FIG. 4 is an orthographic side view of the smoke evacuating surgical device depicting the preferred embodiment showing a non-obstructing cutout of the distal port opening.

FIG. 5 depicts an overall top view of a smoke evacuation surgical device longitudinal axis in relation to three different line of sight angles.

FIG. 6 shows the perspective view of a common prior art distal port having a circular horizon obstructing the surgeon line of sight shown as a dashed line.

FIG. 7 shows the perspective view of an alternate embodiment with multi-planar non-obstructing cutout of a distal port to enhance visibility.

FIG. 8 shows a perspective view of an alternate embodiment having a non-obstructing cutout resulting in a change to the horizon line for enhanced surgeon visibility.

FIG. 9 shows a perspective view of a rapid taper distal port having multiple non-obstructing cutouts resulting in better visibility with at least a plurality of cutouts to improve smoke evacuation flow.

FIG. 10 shows a perspective view of the preferred embodiment of non-obstructing cutout as adjacent contiguous distal port openings with symmetrical cutouts to enhance visibility and maximize smoke evacuation flow.

FIG. 11 is colinear to the surgeon line of sight at a 30-degree acute angle comparing a prior art distal port and the preferred embodiment distal port.

FIG. 12 is colinear to the surgeon line of sight at a 20-degree acute angle comparing prior art distal port and the preferred embodiment distal port.

FIG. 13 is colinear to the surgeon line of sight at a 10-degree acute angle comparing prior art distal port and the preferred embodiment distal port.

### DETAILED DESCRIPTION OF DRAWINGS

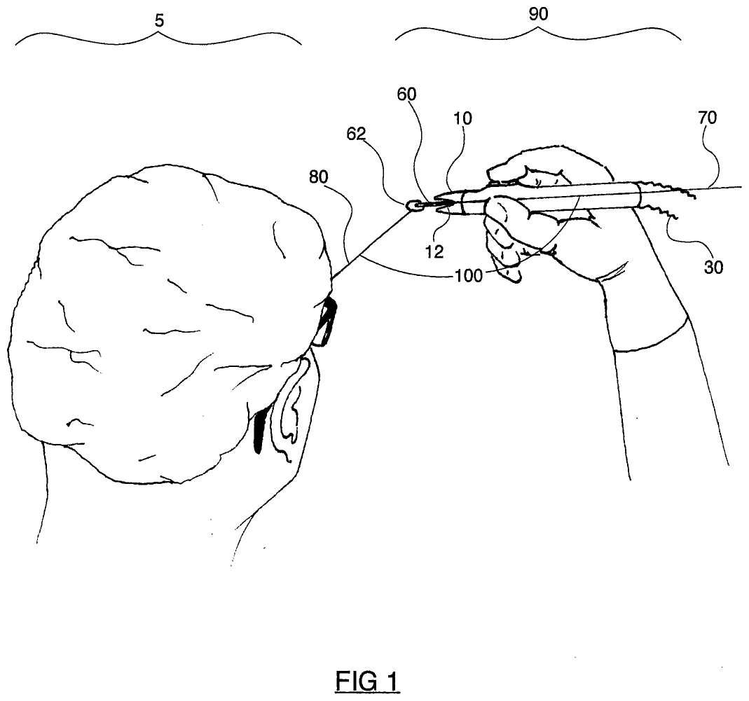

FIG. 1 is a back view of a surgeon 5, holding a smoke evacuating surgical device 90, having a distal port 10 with a non-obstructing cutout 12 and proximal smoke evacuating means 30, depicting an obtuse angle 100 between the surgeon line of sight 80 and the longitudinal axis 70 of the smoke evacuating surgical device 90, showing the optimal unobstructed side visibility of the ‘energy source means’ 60 and ‘energy point of contact’ 62.

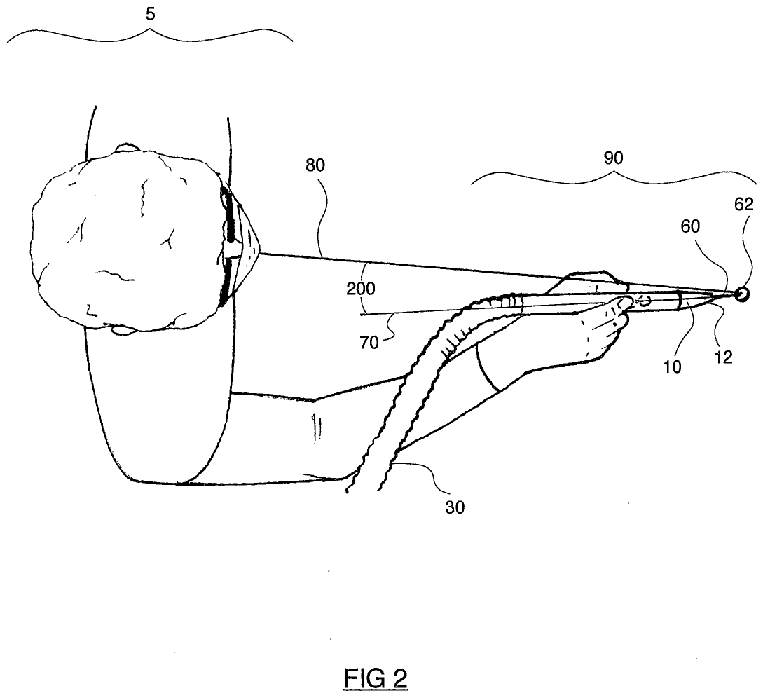

FIG. 2 is a top view of a surgeon 5 holding a smoke evacuating surgical device 90, having a distal port 10, and a proximal smoke evacuating means 30, depicting an acute angle 200 between the surgeon line of sight 80 and the longitudinal axis 70 of the smoke evacuating surgical device 90 having non-obstructing cutouts 12 allowing unobstructed acute visibility of the ‘energy source means’ 60 and ‘energy point of contact’ 62.

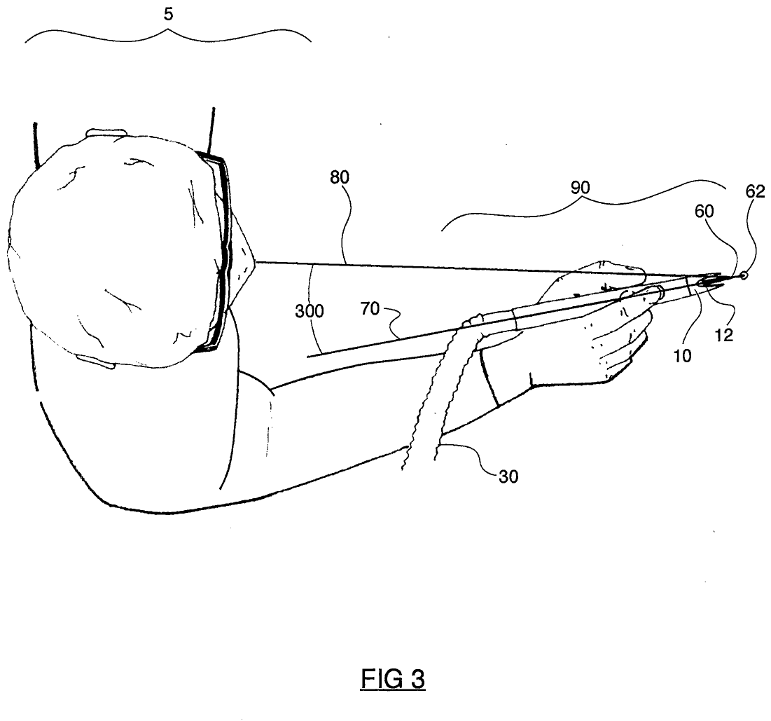

FIG. 3 is a top view of a surgeon 5, holding a smoke evacuating surgical device 90, having a proximal end smoke evacuating means 30, depicting an acute angle 300 between the surgeon line of sight 80 and the longitudinal axis 70 of the smoke evacuating surgical device 90, showing a rotatably distal port 10 aligning non-obstructing cutout 12 to allow various acute angles of unobstructed view of the ‘energy source means’ 60 and ‘energy point of contact’ 62.

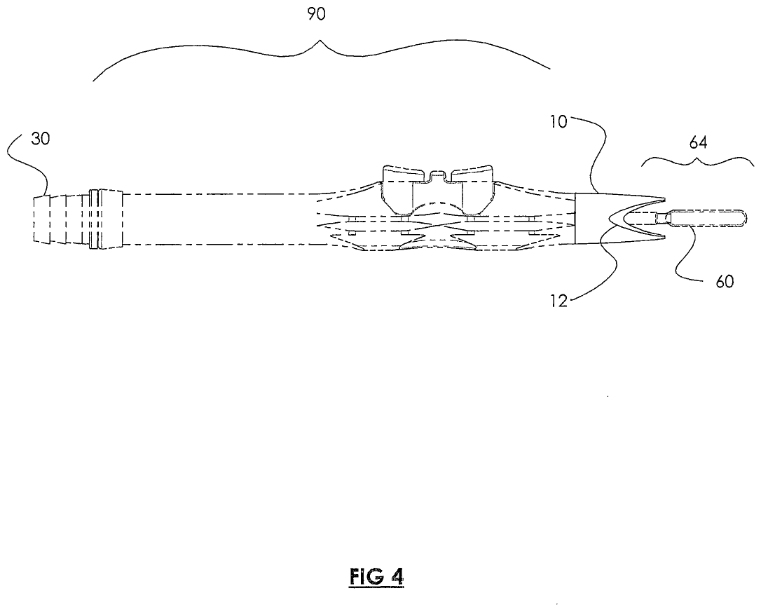

FIG. 4 shows the side view of the preferred embodiment of a smoke evacuating surgical device 90, having a distal port 10 with non-obstructing cutout 12 fully exposing the ‘energy delivery means’ 60 with a working distance 64.

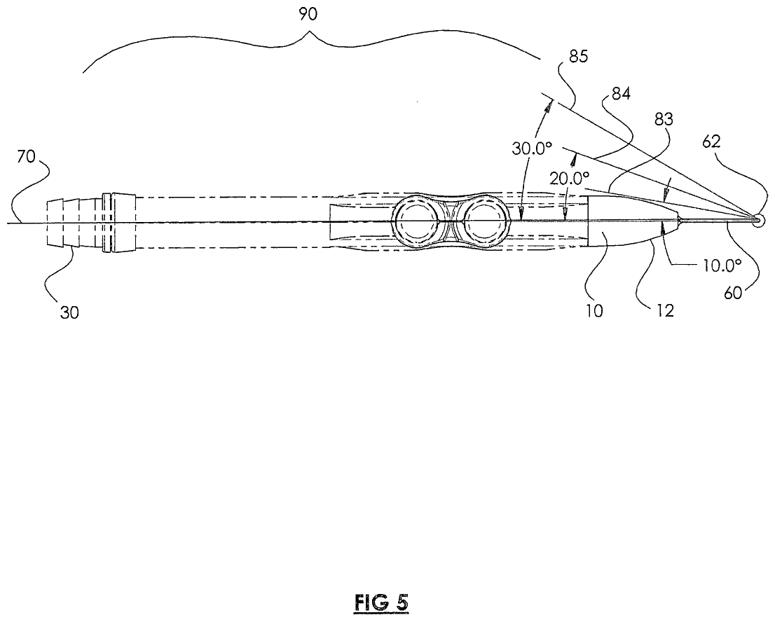

FIG. 5 is a top view depicting various surgeon lines of sight, acute angles 85, 84, 83, of approximately but not limited to 30, 20, 10 degrees respectively to the ‘energy point of contact’ 62, in relation to the position and orientation of the longitudinal axis 70 of the smoke evacuating surgical device 90, and corresponding ‘energy source means’ 60, illustrating the comparison of acute angles 85, 84, 83, as they become smaller.

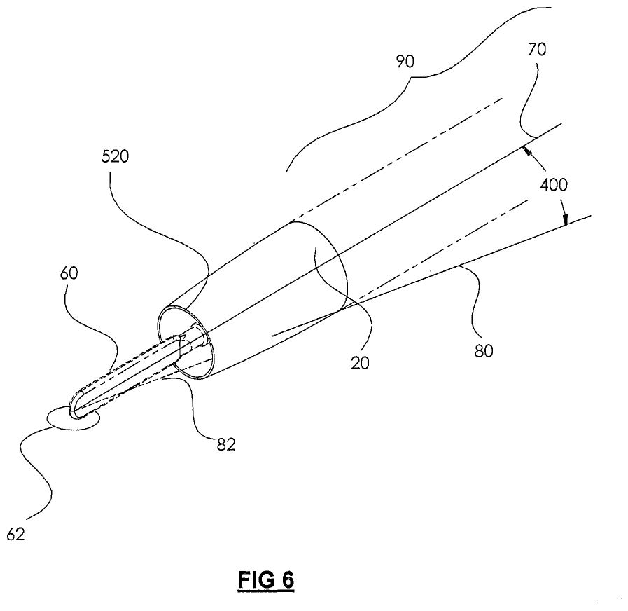

FIG. 6 shows a perspective view of a prior art smoke evacuating device 90 with a common distal port 20, configured as a slightly tapering hollow cylinder, but not limited to tapering, forming a circular horizon 520 to allow optimal suction flow, and an ‘energy source means’ 60 concentric, but not limited to being concentric. The circular obstructing horizon 500, of the distal port 20, prevents an acute visual angle 400 formed by the surgeon's line of sight 80, to the longitudinal axis 70 of the smoke evacuating surgical device 90, to visibly see the ‘energy source means’ 60 and ‘energy point of contact’ 62. The surgeon's line of sight 80 obstruction is colinear to line of sight 80 is non-visible by a visible obstruction shadow shown by a dashed line 82.

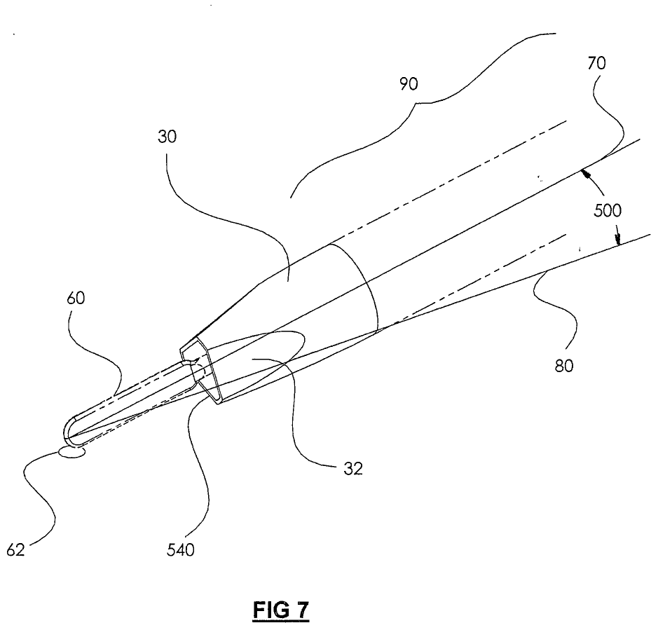

FIG. 7 shows a perspective view of an alternate embodiment of a distal port 30, having multiple but not limited to multiple, non-obstructing cutout surface facets 32 to enhance visibility by lowering the visual obstruction horizon 540 allowing an acute visual angle 500, formed by the surgeon's line of sight 80, longitudinal axis 70 of the smoke evacuating surgical device 90 to view the ‘energy source means’ 60 and ‘energy point of contact’ 62.

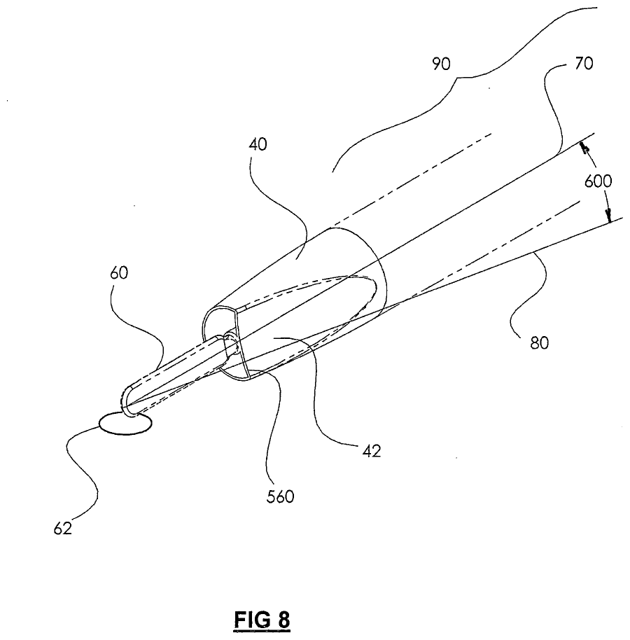

FIG. 8 shows a perspective view of an alternate embodiment having a non-obstructing cutout 42 on distal port 40, but not limited to at least one non-obstructing cutout 42 non-concentric to the longitudinal axis 70 of the smoke evacuating surgical device 90 to increase smoke evacuation flow and enhance the surgeons' line of sight 80 at acute angles 600.

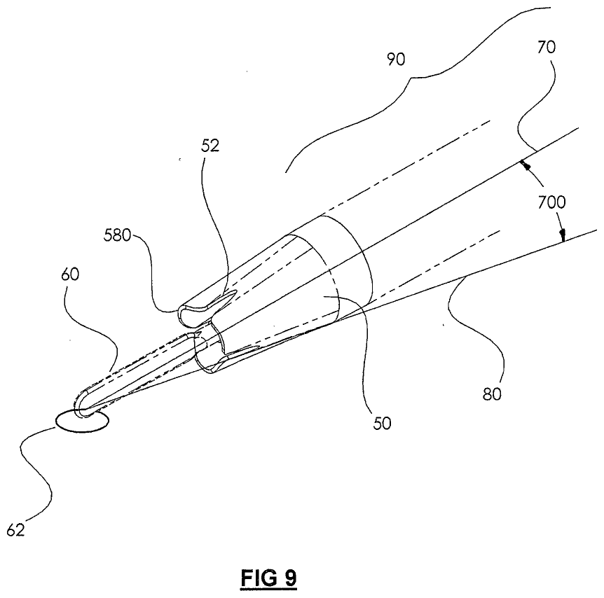

FIG. 9 shows perspective view of an alternate embodiment distal port 10 to enhance visibility, having symmetrical, but not limited to symmetrical, non-obstructing cutout 52 to improve suction flow while creating an irregular horizon line 580, by removing material from the distal port 50, to enhance the acute angle 700 visibility of the surgeon's line of sight 80, to the smoke evacuating surgical device longitudinal axis 70, and ‘energy source means’ 60 and ‘energy point of contact’ 62.

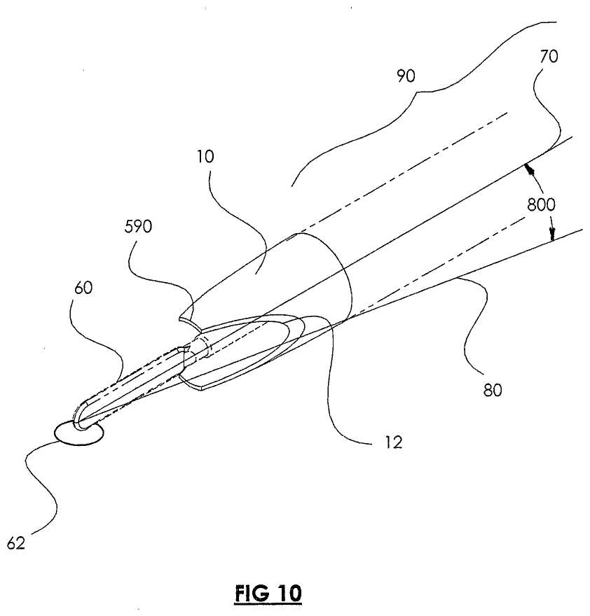

FIG. 10 shows perspective view of the preferred embodiment distal port 10 to enhance visibility, having symmetrical, but not limited to symmetrical, non-obstructing cutouts 12 to improve suction flow while creating an irregular edge opening 12, by removing material from the distal port horizon 590, to enhance the acute angle 800 visibility of the surgeon's line of sight 80, to the smoke evacuating surgical device longitudinal axis 70, and ‘energy source means’ 60 and ‘energy point of contact’ 62.

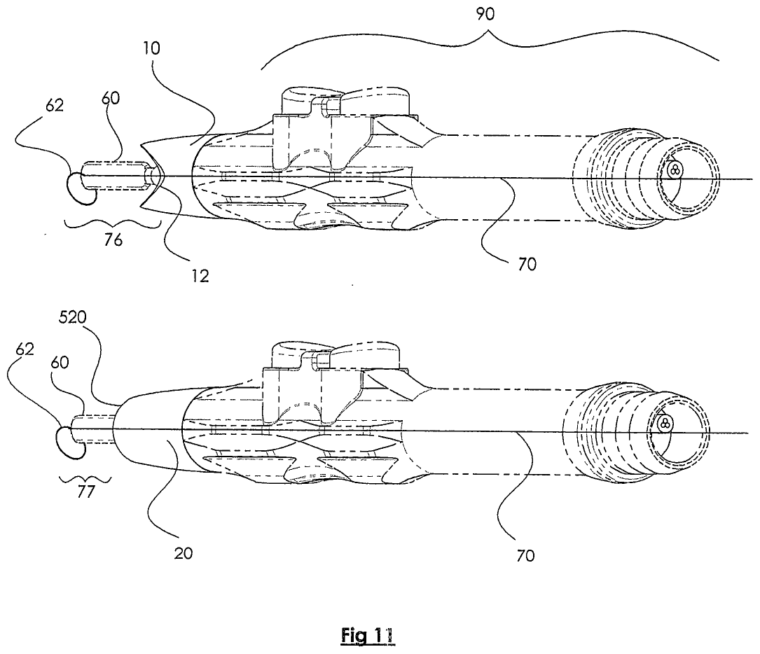

FIG. 11 depicts the preferred embodiment as viewed colinear to the surgeons' line of sight 82 with an acute angle of approximately but not limited to 30 degrees showing the ‘energy point of contact’ 62, in relation to the position and orientation of the longitudinal axis 70 of the smoke evacuating surgical device 90, and corresponding ‘energy source means’ 60, to illustrate the comparison in visibility of the working distance 76 against the lower prior art working distance 77 to the preferred embodiment.

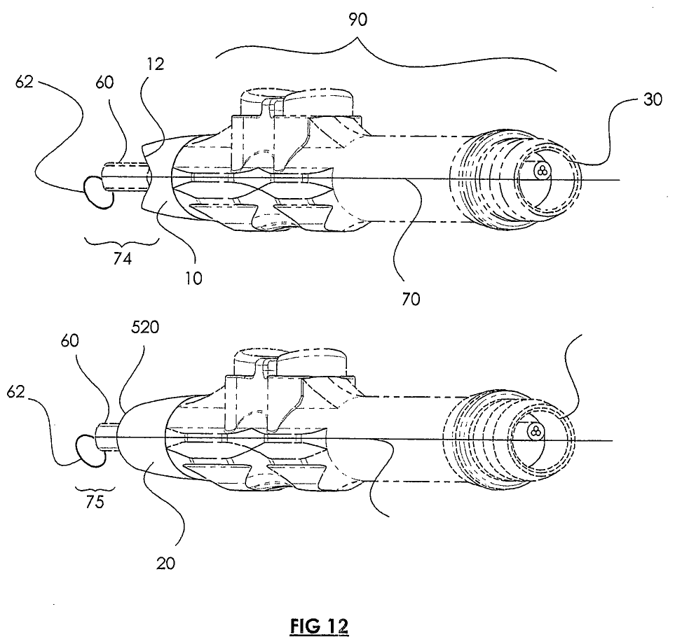

FIG. 12 depicts the preferred embodiment as viewed colinear to the surgeons' line of sight 82 with an acute angle of approximately but not limited to 20 degrees showing the ‘energy point of contact’ 62, in relation to the position and orientation of the longitudinal axis 70 of the smoke evacuating surgical device 90, and corresponding ‘energy source means’ 60, to illustrate the comparison in visibility of the working distance 74 against the lower prior art working distance 75 to the preferred embodiment.

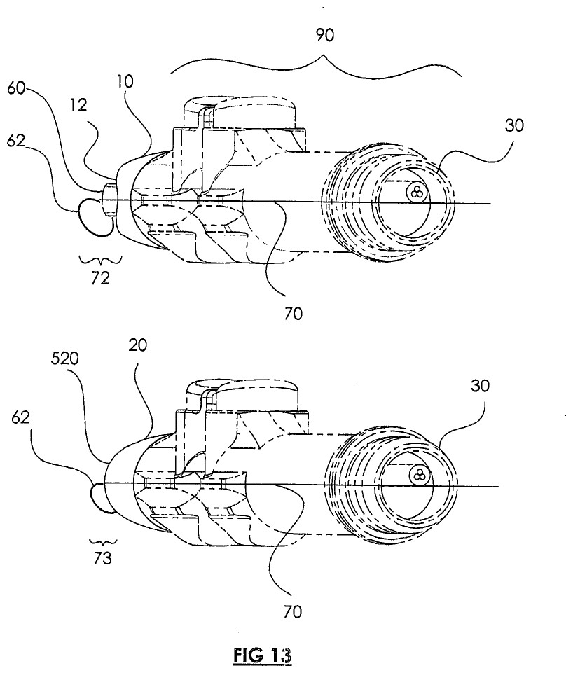

FIG. 13 depicts the preferred embodiment as viewed colinear to the surgeons' line of sight 82 with an acute angle of approximately but not limited to 30 degrees showing the ‘energy point of contact’ 62, in relation to the position and orientation of the longitudinal axis 70 of the smoke evacuating surgical device 90, and corresponding ‘energy source means’ 60, to illustrate the comparison in visibility of the working distance 72 against the lower prior art working distance 73 to the preferred embodiment.

What is claimed:

1. A distal end configuration of a smoke evacuating surgical device comprised of:
a distal port having at least one non-obstructing cutout,
emanating away from said distal opening,
to allow surgeon visibility at acute angles,
of the energy source means and the energy point of contact.

2. A distal end configuration of claim 1 having at least one non-obstructing cutout as a surface change lowering the distal port horizon allowing said surgeon visibility at said acute angles.

3. A distal end configuration of claim 1 wherein said at least one non-obstructing cutout is the material removal of said distal port allowing said surgeon visibility at said acute angles.

4. Said distal end configuration of claim 1 having a selectively rotatable distal port to orient said non-obstructing cutout to align with said surgeons' line of sight of said energy source means and the energy point of contact.
